(19) United States
(12) Patent Application Publication
(10) Pub. No.: US 2004/0217146 A1
(43) Pub. Date: Nov. 4, 2004

(54) Surgical stapler apparatus and method

(76) Inventor: Joachim Beck, Sennwald (SZ)

Correspondence Address: Lisa J. Moyles, United States Surgical, a division of TYCO, Healthcare Group LP, 150 Glover Avenue, Norwalk, CT, 06856, US

(21) Appl. No.: 10/858,737

(22) Filed: Jun. 2, 2004

(30) Foreign Application Priority Data
Dec. 20, 2002 (WO) PCT/US02/41127

(51) Int. Cl. A61B 17/10; A61B 17/04

(52) U.S. Cl. 227/176.1; 227/180.1; 606/142

(57) ABSTRACT

The present disclosure relates to circular staplers for performing rectal mucosectomies and other surgical procedures. The circular staplers include a shaft having a proximal and a distal end, a cartridge assembly having a plurality of staples and operatively coupled to the distal end of the shaft, the cartridge assembly having a housing and a distal end defining an inner chamber, an anvil assembly operatively coupled to the cartridge assembly, and a channel adapted to communicate with a source of vacuum and the cartridge assembly to transmit a vacuum to the inner chamber of the cartridge assembly for drawing tissue into the inner chamber of the cartridge assembly.

### CROSS-REFERENCE TO RELATED APPLICATIONS

The present application claims priority of U.S. Provisional Patent Application Serial No. 60/342,672 filed Dec. 20, 2001, the entire contents of which are incorporated herein by reference.

### BACKGROUND

1. Technical Field

The present disclosure relates to surgical instruments, e.g., surgical fastener or stapler apparatus and, more particularly to circular surgical staplers for performing surgical procedures, including but not limited to rectal mucosectomies, rectal anopexies, anastamoses, hemorrhoidectomies, or the like. In addition the present disclosure relates to methods of performing such surgical procedures.

2. Background of Related Art

Various types of surgical fasteners or staplers are known for the application of fasteners or staples to tissue in order to join adjacent tissue. For example, it is known to use various types of staplers in gastric and esophageal surgery, for example in both classic or modified gastric reconstructions performed end-to-end, end-to-side, or side-to-side, as well as for performing a hemorrhoidectomy.

Hemorrhoids are a swollen twisted mass of blood vessels that are located just inside the anus and which are caused by chronic straining for example from constipation and/or childbirth. The plexus of vascular tissue beneath the epithelial lining of the anal canal is called the corpus cavernosus recti or anal cushion. These plexus connect arteries to veins without intervening capillaries thus creating the vascular component of the anal cushions. These arteriovenous channels control the size of the anal cushions by regulating the blood volume flowing through them. Chronic straining damages the submucosal fibroelastic connective tissue and the anal cushions protrude into the anal canal and produce painless bleeding (first degree hemorrhoids). Only prolapsed hemorrhoids lying outside the anal canal may become strangulated by the internal sphincter activity with possible thrombosis of the venous plexus (fourth-degree hemorrhoids). This condition may result in gangrene with the risk of abscess formation.

Hemorrhoids can be of two types, external and internal. A ribbed dentate line is located about 2.5 to 3 cm in from the exterior of the anus and marks the change from the anus to the rectum. Hemorrhoids are found in the anal area below this line. Internal hemorrhoids generally are found in the rectal area below this line and external hemorrhoids generally are external of the anal ring. Internal hemorrhoids are generally formed from arteriovenous anastomoses or connections that reside in a submucosal space within the wall of the rectum, approximately 2.5 to 5 cm in from the exterior of the anus. Due to its proximity to the anus, internal hemorrhoids can protrude from the wall of the rectum in either one localized area, more than one localized area, or circumferentially around the rectum and in certain severe cases, the internal hemorrhoids can protrude out of the anus.

A wide variety of surgical methods have been suggested for the treatment of severe internal hemorrhoids. One method is a closed hemorrhoidectomy. According to this method a retractor is inserted into the anus to obtain access to a hemorrhoidal site. The surgeon then clamps the hemorrhoid with alligator clamps, ligates the vessels, and dissects the hemorrhoid from the rectal wall with a scalpel or scissors. Once the hemorrhoid is removed, the surgical site is sutured closed. The retractor is then rotated to another position and the remaining hemorrhoids are dealt with in a similar manner until all of the hemorrhoids have been removed.

Another method for the removal of internal hemorrhoids is an open hemorrhoidectomy. According to this procedure, rather than using a retractor, the anus is gently dilated with two fingers and forceps are placed at the mucocutaneous junction of each primary hemorrhoid. The hemorrhoids are pulled down and a second forceps is applied to the main bulk of each hemorrhoid to produce “a triangle of exposure”. Next, the clamped, hemorrhoid is dissected from the sphincter muscle and is dissected proximally as far as the pedicles and then ligated or tied. Unlike the closed procedure, the wound is not sutured closed, but is left open with a light dressing applied to the wound.

Yet another hemorrhoidectomy procedure involves excising hemorrhoidal tissue above the dentate line and excising and suturing the redundant rectal mucosa to the anoderm. This procedure is particularly used in conjunction with circumferential hemorrhoids.

In a paper by Dr. G. Allegra entitled “Particular Experience with Mechanical Sutures: Circular Stapler for Hemorrhoidectomy,” presented to the 1.sup.st. National Conference of the Italian Viscerosynthesis Association in May 28-30, 1989, and published in GIORN Chir. Vol. 11-No. 3-pp 95-97, March 1990, Dr. Allegra disclosed a simpler and faster method for performing a hemorrhoidectomy. The paper discloses the use of a conventional circular stapler to perform a hemorrhoidectomy on second and third degree hemorrhoids.

According to the procedure detailed by Dr. Allegra, three fingers are used to dilate the anus and to place a continuous submucosal circle of sutures at the base of the pectinate or dentate line. Next, a conventional circular stapling instrument, having a stapling end effector, is opened by extending an anvil assembly away from a stapling head assembly. The opened stapling end effector is placed into the anus of a patient and positioned such that the anvil assembly of the stapling end effector is distal of the suture ring and the stapling head assembly (of the stapling end effector) is proximally outside of the patient. This placement enables the surgeon to reach within the anus in order to grasp the loose ends of the suture. With the stapling end effector in place, the loose ends of the suture are drawn out of the anus and out of the stapling end effector between the open anvil and the stapling head assembly. The loose ends of the suture are then pulled to draw the circle of suture closed and to draw the hemorrhoidal tissue in around the anvil shaft connecting the open anvil to the stapling head assembly of the circular stapling instrument. Next, the surgeon tightly knots the suture about the anvil shaft and closes the anvil upon the hemorrhoidal mass. The loose ends of the suture protrude from the stapling end effector between the closed anvil and the stapling head assembly. The stapler is fired to perform the hemorrhoidal transection. Once fired, the circular stapling instrument is removed from the anus with the transected hemorrhoids captured within.

One limitation of Dr. Allegra's procedure is the depth that the circular stapler can be placed into the anus. As described above, the open anvil assembly of the stapling end effector is placed distally of the suture ring and the stapling head assembly is placed proximally outside of the patient thereby enabling the surgeon to grasp the loose ends of the suture and to draw the suture out of the anus through the gap between the anus and the stapling head assembly. The gap, between the anus and the stapling head assembly, is needed to withdraw the suture from the anus and thus limits the depth that the stapling end effector can be placed into the anus. If the hemorrhoids are located deeper into the anal canal, as in the case of internal hemorrhoids, the stapling head assembly enters the anus and effectively blocks the surgeon from accessing the loose ends of the suture.

An additional limitation of Dr. Allegra's procedure is the amount of hemorrhoidal tissue that can be drawn into the stapling end effector of a conventional circular stapling instrument. Hemorrhoids are drawn into and around an anvil shaft (connecting the open anvil assembly to the stapling head assembly) by tightening a continuous loop of suture placed below the dentate line. This action draws the hemorrhoids around the anvil shaft but does not draw the hemorrhoids into the inner chamber of the stapling head assembly. This limits the amount of hemorrhoidal tissue that can be brought into the stapling end effector and the surgeon may remove only part of a hemorrhoid.

In U.S. Pat. No. 6,083,241, to Longo et al. issued Jul. 4, 2000, a method for removing internal hemorrhoids from a patient using a circular stapler is described. The method according to Longo, et al. provides the surgeon with the improved ability to draw purse sutured hemorrhoids around an anvil shaft and into the inner chamber of the stapling head assembly and which method provides the surgeon with the ability to remove hemorrhoids which are deep within the anus.

A suture is placed above the internal hemorrhoids of the patient. The stapling end effector is inserted into the anus of a patient with the anvil assembly in the open position and is positioned such that, according to Longo, the purse string suture is located usually 3-4 cm above the dentate line and therefore above the hemorrhoids. The purse string suture is brought around, between the stapling head assembly and the anvil assembly and loosely tied. The ends of the suture are then spread apart, pulled and secured. A needle hook is employed to grasp the suture. The hook is used to withdraw the suture through a passageway in the stapling head assembly and out of the casing of the stapling head assembly. The suture is pulled so as to draw the hemorrhoidal tissue inwardly around the anvil shaft and the interior drive shaft and into the chambers of the anvil and cartridge. The anvil assembly is moved from the open position to the closed position so as to clamp the hemorrhoidal tissue therebetween. The circular stapling instrument is then fired in order to staple and cut the hemorrhoidal tissue and the stapling instrument is removed from the patient to remove the tissue from the patient.

A significant drawback of both the Dr. Allegra and the Longo et al. procedures is the complexity of the procedures. Each calls for the hemorrhoidal tissue to be sutured, prior to stapling and cutting. This significantly increases the time of performing the procedure and in turn increases the potential for complications and the potential for trauma to the patient. As described above, each procedure generally requires that a purse string type suture be first sewn into the tissue by using a retractor to expose a portion of the tissue; suturing the exposed portion of the tissue; repositioning the retractor adjacent the sutured portion and repeating these steps around the circumference of the rectal cavity until the entire tissue has been sutured.

Thus, the need exists for surgical staplers and/or apparatus which are less complex and faster to use than existing surgical staplers. In addition, the need exists for staplers and/or apparatus which reduce the trauma experienced by the patient during the surgical procedures described above. Further, the need exists for methods of performing the above described surgical procedures which are less complex and less time consuming than existing methods of performing the same.

### SUMMARY

The present disclosure is directed to apparatus for performing surgical procedures including rectal mucosectomies or rectal anopexies to treat, for example, hemorrhoids and/or rectal mucosal prolapse. The present disclosure is also directed to methods of using the apparatus of the present disclosure to perform the above and other procedures.

In accordance with one aspect of the present disclosure, a surgical instrument for performing the above procedures and other procedures includes a shaft having a proximal end and a distal end, a cartridge assembly including a plurality of fasteners and operatively coupled to the distal end of the shaft, the cartridge assembly having a housing and a distal end, and defining an inner chamber, at least one opening formed in the surgical instrument, the opening extending through the exterior of the instrument and being in communication with the inner chamber and adapted to communicate with a vacuum source, an anvil assembly operatively coupled to the cartridge assembly and disposed opposite the cartridge assembly, the cartridge assembly and the anvil assembly being configured and adapted to fire and secure fasteners into target rectal mucosal tissue, and a vacuum channel extending through the cartridge assembly and in communication with the inner chamber such that when a vacuum is applied through the channel, the vacuum is transmitted into the inner chamber of the cartridge assembly to draw rectal mucosal tissue into the inner chamber.

In one embodiment, the at least one opening is in the exterior of the housing. Preferably, the at least one opening is located proximally of the cartridge assembly. In another embodiment, the at least one opening extends from the shaft to the inner chamber.

It is envisioned that the vacuum is drawn into the distal end of the cartridge assembly. Preferably, the vacuum channel transmits the vacuum through the at least one opening to draw rectal mucosal tissue from areas surrounding and/or above the distal end of the cartridge assembly into the inner chamber of the cartridge assembly.

It is contemplated that the cartridge assembly includes an annular blade disposed therewithin, radially inward of the fasteners. In one embodiment, the annular blade includes a peripheral wall and an interior chamber radially inward of the wall, wherein the vacuum is transmitted to an area within the interior chamber of the annular blade to draw tissue surrounding and/or above the distal end of the cartridge assembly into the interior chamber. In another embodiment, the annular blade has a distal end and an open proximal end, and the vacuum is transmitted axially through the proximal end. In yet another embodiment, the annular blade has a distal end, a proximal end, and a bottom wall at the proximal end, the bottom wall having at least one opening therethrough for drawing of the vacuum axially therethrough.

It is envisioned that the vacuum chamber extends within and along a length of the shaft. It is further envisioned that the outer surface of the shaft includes gradations arranged axially along a length thereof to allow a user to visually determine a distance from the distal end of the cartridge assembly to the anal ring of the patient when the distal end of the cartridge assembly is inserted into the anal ring of the patient. Preferably, the gradations are sufficient to determine a distance of at least about 5 to 6 cm from the distal end of the cartridge assembly to the anal ring of the patient.

A method of performing a rectal mucosectomy is disclosed. The method includes the steps of providing a circular fastener apparatus having a distal end and including a shaft, a cartridge assembly operatively coupled to the distal end of the shaft and having a plurality of fasteners and an inner chamber, an anvil assembly operatively coupled to the cartridge assembly and supported at the distal end of the shaft adjacent the cartridge assembly, and a vacuum application element configured and adapted to transmit a vacuum to the inner chamber of the cartridge assembly of the circular fastener apparatus.

The method further includes the steps of inserting a distal end of the circular fastener apparatus distally into the rectum of a patient such that the distal end of the cartridge assembly is located at or distally of hemorrhoidal tissue, providing a gap between the upper border of the cartridge assembly and anvil assembly, applying a vacuum to the cartridge assembly such that rectal mucosal tissue is drawn into the inner chamber of the cartridge assembly, approximating the anvil and cartridge assemblies to one another, and firing the circular fastener apparatus to sever the rectal mucosal tissue from the rectal wall of the patient.

It is envisioned that the distal end of the cartridge assembly is positioned distally of the hemorrhoidal tissue prior to the application of the vacuum. Preferably, the anvil and cartridge assemblies are approximated with one another when the circular fastener apparatus is inserted into the rectum, and they are separated from one another prior to application of the vacuum.

Preferably, the distal end of the circular fastener apparatus is inserted into the rectum of the patient such that the distal end of the cartridge assembly is positioned about 3 to 4 cm distally of the dentate line. It is envisioned that the anvil assembly is spaced from the cartridge assembly about 2 to 3 cm prior to application of the vacuum. It is further envisioned that the vacuum is drawn axially through the cartridge assembly.

In another aspect of the disclosure a circular stapler is disclosed. The circular stapler includes a shaft having a proximal and a distal end, a cartridge assembly having a plurality of staples and operatively coupled to the distal end of the shaft, the cartridge assembly having a housing and a distal end defining an inner chamber, an anvil assembly operatively coupled to the cartridge assembly, and a channel adapted to communicate with the vacuum source and the cartridge assembly to transmit a vacuum to the inner chamber of the cartridge assembly for drawing tissue into the inner chamber of the cartridge assembly.

It is envisioned that the cartridge assembly includes an annular blade disposed within the inner chamber thereof. Preferably, the vacuum is transmitted internally of the annular blade. The annular blade has an interior chamber and is preferably disposed radially inside of the fasteners, such that by the vacuum draws surrounding tissue into the interior chamber of the annular blade.

In another aspect, a circular stapler is disclosed including a shaft having a proximal end and a distal end, a cartridge assembly including a plurality of staples and operatively coupled to the distal end of the shaft, the cartridge assembly including at least one opening and defining an inner chamber, the at least one opening extending from the exterior of the apparatus through to the inner chamber, an anvil assembly supported adjacent the cartridge assembly and movable relative to the cartridge assembly between spaced and approximated positions, and a vacuum collar configured and adapted to be couplable to at least the cartridge assembly such that the at least one opening formed in the cartridge assembly is covered by the vacuum collar, such that when the vacuum collar is applied to at least a portion of the cartridge assembly, and a vacuum is applied to the vacuum collar, the vacuum is transmitted through the at least one opening to the inner chamber of the cartridge assembly to draw tissue into a gap defined between the cartridge and anvil assemblies.

Preferably, the vacuum collar creates a fluid tight seal around the cartridge assembly and the shaft. The vacuum collar defines a vacuum chamber around at least a portion of the shaft and of the cartridge assembly. The vacuum collar includes at least one vacuum transmitting lumen for transmitting the vacuum to the vacuum chamber. It is envisioned that the vacuum collar includes a pair of semi-cylindrical cuffs which are couplable to one another by fastening elements. Preferably, the vacuum collar transmits the vacuum through the at least one opening formed in the cartridge assembly to draw tissue surrounding and distally of a distal end of the cartridge assembly into the inner chamber of the cartridge assembly.

In one aspect of the disclosure, the cartridge assembly includes an annular blade located radially inward of the fasteners. Accordingly, the vacuum is transmitted to an area within the annular blade to draw tissue surrounding the cartridge assembly into the annular blade. The surgical apparatus preferably further includes a source of vacuum fluidly and operatively coupled to the vacuum collar. A valve is preferably fluidly coupled to the source of vacuum, via a conduit, wherein the valve controls the transmission of vacuum to the vacuum collar. It is contemplated that the vacuum chamber extends substantially along the entire length of the shaft.

In another aspect of the disclosure there is provided a vacuum collar for use with a circular stapler of the type including an elongated shaft having a distal end supported adjacent the cartridge assembly and coupled to a distal end of the shaft and an anvil assembly supported adjacent the cartridge assembly. The vacuum collar preferably includes a pair of cuffs operatively couplable to one another, each of the pair of cuffs being configured and adapted, when coupled to the circular stapler, to surround at least a portion of the shaft and/or the cartridge assembly and to provide a passageway to the cartridge assembly, and at least one connection for a vacuum conduit to operatively and fluidly couple the conduit to at least one of the pair of cuffs, the at least one vacuum conduit being for transmitting a vacuum to the vacuum collar and through the passageway to draw a vacuum within the cartridge assembly.

The vacuum collar defines a vacuum chamber surrounding at least a portion of the shaft and/or the cartridge assembly when the vacuum collar is coupled to the circular stapler. Each cuff is preferably semi-cylindrical and includes a pair of longitudinal terminal edges, a distal edge configured and dimensioned to contact and seal an outer surface of the cartridge assembly, and a proximal edge configured and dimensioned to contact and seal an outer surface of the shaft. It is envisioned that the longitudinal, distal and proximal edges of each cuff includes a sealing element. Preferably, each longitudinal terminal edge includes a tab extending radially outward therefrom, each tab including an aperture formed therein for receiving a fastening element therethrough, wherein the tabs from one cuff align with the tabs from the other cuff. It is envisioned that the vacuum collar extends along substantially the entire length of the shaft.

The present disclosure also provides a method of performing a rectal mucosectomy. The method includes the steps of providing a circular stapler including a shaft having a distal end, a cartridge assembly having an inner chamber and a cutting blade, and operatively coupled to the distal end of the shaft, an anvil assembly operatively coupled to the cartridge assembly and supported adjacent to the cartridge assembly, and a vacuum application element configured and adapted to transmit a vacuum to the inner chamber of the cartridge assembly of the circular stapler.

The method further includes the steps of inserting a distal end of the circular stapler into the rectum of a patient such that a portion of the cartridge assembly is located above the dentate line, applying a vacuum through the channel to the inner chamber of the cartridge assembly, such that the rectal mucosal tissue is drawn into the inner chamber of the cartridge assembly, approximating the anvil and cartridge assemblies to one another, and firing the circular stapler to sever the rectal mucosal tissue from the patient and anastomose the remaining tissue.

Preferably, the anvil assembly is positioned proximally of and/or above the dentate line prior to the application of the vacuum. It is contemplated that the anvil and cartridge assemblies are approximated with one another prior to insertion into the rectum and separated from one another for application of the vacuum. It is envisioned that the vacuum application element is either a vacuum channel and/or a vacuum collar.

In accordance with a further aspect of the present disclosure, the circular stapler for performing a rectal mucosectomy includes a shaft having a proximal and a distal end, a cartridge assembly containing a plurality of staples and operatively coupled to the distal end of the shaft and defining an inner chamber therein, an anvil assembly operatively coupled to the cartridge assembly, the cartridge assembly and the anvil assembly being configured and adapted to fire and secure a plurality of staples into a target site, and a vacuum channel operatively and fluidly couplable to the cartridge assembly to transmit a vacuum to the inner chamber of the cartridge assembly, independent of the anvil assembly, for drawing distal and/or surrounding tissue into the internal chamber of the cartridge assembly.

It is contemplated that the cartridge assembly includes an annular blade disposed within the internal chamber thereof and wherein the vacuum is transmitted internally of the annular blade. Preferably, the annular blade is disposed radially inside of the fasteners. Accordingly, the vacuum draws tissue into the channel defined by the annular blade.

In accordance with yet another aspect of the disclosure, the circular stapler for performing a surgical procedure includes a shaft having a proximal end and a distal end, an anvil retainer assembly, a circular cartridge assembly including a plurality of staples and operatively coupled to the distal end of the shaft, the cartridge assembly having a housing and a distal end, and defining an inner chamber, an anvil assembly operatively coupled to the cartridge assembly and disposed opposite the cartridge assembly, the cartridge assembly and the anvil assembly being configured and adapted to fire and secure staples into target tissue, and a vacuum channel extending externally of the anvil retainer assembly, through the cartridge assembly, and in communication through the channel, a vacuum is transmitted through the channel axially into the inner chamber of the cartridge assembly to draw tissue into the inner chamber.

It is envisioned that the surgical procedure is one of a circular stapler anopexy, a hemorrhoidectomy, a rectal mucosectomey, and an anastomoses.

It is further envisioned that in either of the methods disclosed herein, the vacuum is applied at a pressure of at least 8-10 bars.

Other objects and features of the present disclosure will become apparent from consideration of the following description taken in conjunction with the accompanying drawings.

### BRIEF DESCRIPTION OF THE DRAWINGS

By way of example only, preferred embodiments of the disclosure will be described with reference to the accompanying drawings, in which:

FIG. 1 is a perspective view of a conventional circular surgical stapling apparatus;

FIG. 2 is an enlarged perspective view of the distal end of the stapling apparatus shown in FIG. 1 with a vacuum collar, in accordance with the present disclosure, shown operatively coupled thereto;

FIG. 3 is an enlarged exploded perspective view of the vacuum collar shown in FIG. 2;

FIG. 4 is a partial cross-sectional view, taken substantially along the longitudinal axis, of the distal end of the stapling apparatus of FIG. 2 with the vacuum collar shown in FIG. 2 operatively coupled thereto;

FIG. 5 illustrates the anvil assembly of the surgical stapling apparatus of FIG. 1 as it would appear in the open position and vacuum collar of FIG. 3 as the apparatus would appear in an open position inserted through the anus and into the rectum of a patient such that the distal end of the anvil assembly is located distal of the hemorrhoidal tissue and a portion of the cartridge assembly is located proximal of the hemorroidal tissue;

FIG. 6 is similar to FIG. 5 illustrating the application of a vacuum by the vacuum collar to draw the rectal mucosal tissue into the cartridge assembly of the surgical stapling apparatus and the approximation of the anvil assembly to the cartridge assembly;

FIG. 7 is similar to FIG. 5 illustrating the firing of the surgical stapling instrument in order to place a plurality of staples into the submucosal layer and to sever the rectal mucosal tissue from the patient radially inward of the staples;

FIG. 8 illustrates the distal end of a surgical stapling apparatus including an alternative vacuum drawing device coupled to the apparatus; and

FIG. 9 illustrates the distal end of a surgical stapling apparatus according to an alternate embodiment of the present disclosure.

### DETAILED DESCRIPTION OF PREFERRED EMBODIMENTS

Preferred embodiments of the presently disclosed surgical apparatus will now be described in detail with reference to the drawing figures wherein like reference numerals identify similar or identical elements. In the drawings and in the description which follows, the term “proximal”, as is traditional will refer to the end of the surgical apparatus which is closest to the operator, while the term “distal” will refer to the end of the device which is furthest from the operator. While the following description of the surgical apparatus and method will relate mainly to the removal of rectal mucosal tissue from a patient, it is envisioned that the apparatus according to the present disclosure is not limited to the removal of the rectal mucosal tissue and can be used to perform additional or other surgical procedures.

In the present disclosure, the term “rectal mucosectomy” is understood to include rectal-cuff-mucosectomy, circular stapler anopexy, hemorrhoidectomy, anastomosis, colonectomy and any other surgical procedure involving the mucosal wall or other wall of a lumen of the body. The term fastener or fasteners include single or multi-part, e.g., two-part surgical fasteners, surgical staples, and the like. The fasteners can be made or comprised of any suitable material, for example, biocompatible or bioabsorbable.

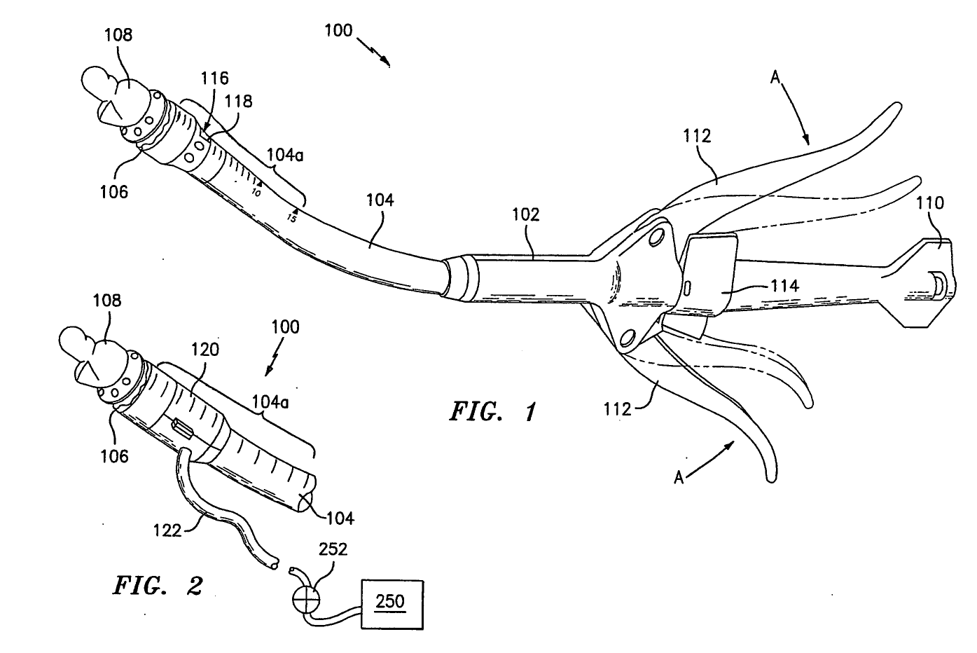

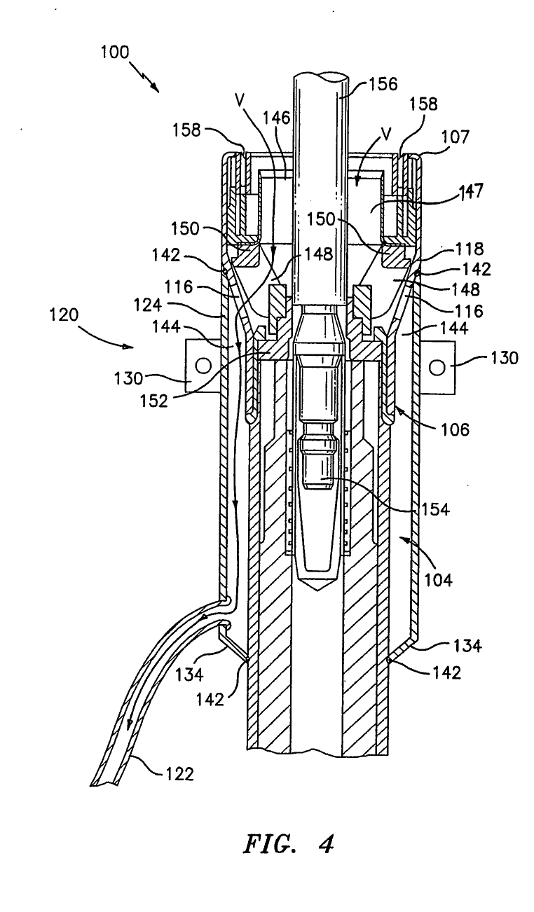

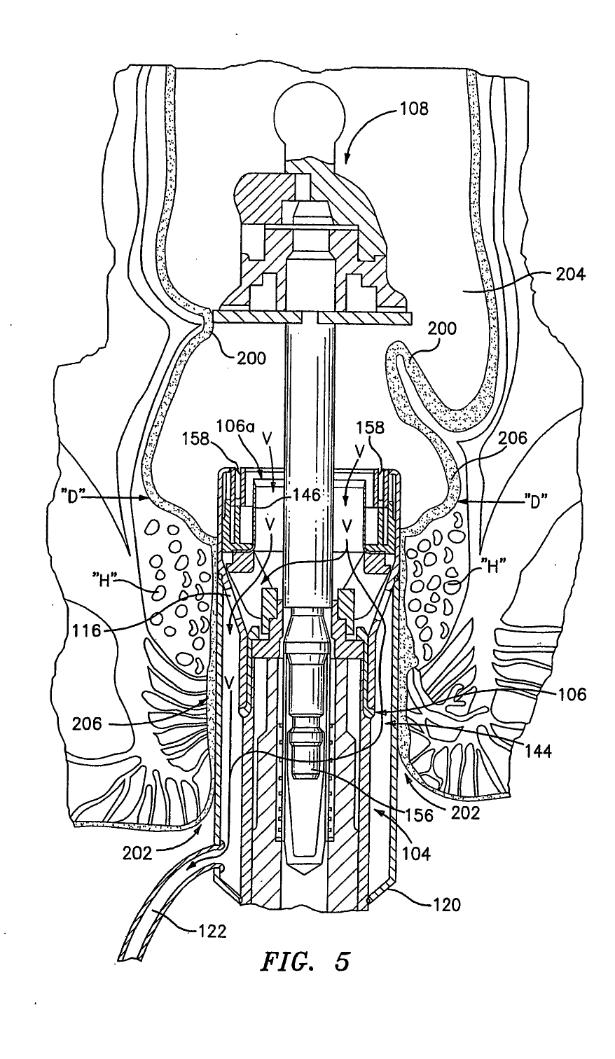

Referring initially to FIG. 1, a surgical stapling apparatus in accordance with the present disclosure is generally designated as 100. Surgical stapling apparatus 100 is of a generally conventional structure and includes a yoke 102, a pusher tube assembly 104 extending from yoke 102 having a housing 107 configured and adapted to carry a cartridge assembly 106 therein, cartridge assembly 106 defining an internal chamber 106a (See FIGS. 4, 5 and 8) at a distal end thereof, and an anvil assembly 108 at a distal end of the apparatus 100. Apparatus 100 has an actuating mechanism (not shown) extending proximally from anvil assembly 108 through pusher tube assembly 104 to a wing nut 110 at a proximal end of apparatus 100, for moving anvil assembly 108 against cartridge assembly 106. In addition, a pair of handles 112 are mounted to apparatus 100, to be manually grasped by the surgeon and moved towards each other as indicated by arrows “A”, in order to fire staples 158 (see FIG. 5) from cartridge assembly 106 towards anvil assembly 108 and to actuate an annular blade 146 (see FIG. 4). A safety lock 114 is also mounted on the proximal end of yoke 102 in order to prevent inadvertent movement of handles 112 towards each other and premature firing of staples 158 or actuating of annular blade 146 (see FIG. 4). As is known in the art, one or more suitable venting holes 116 are provided in a conical section 118 of cartridge assembly 106 for venting the interior of cartridge assembly 106. In accordance with the present disclosure, these and/or other vent holes or apertures can be employed or provided for providing a vacuum to the interior of cartridge assembly 106. A typical surgical stapling apparatus is described in commonly assigned U.S. Pat. No. 5,915,616 to Viola et al., the entire disclosure of which is incorporated herein by reference.

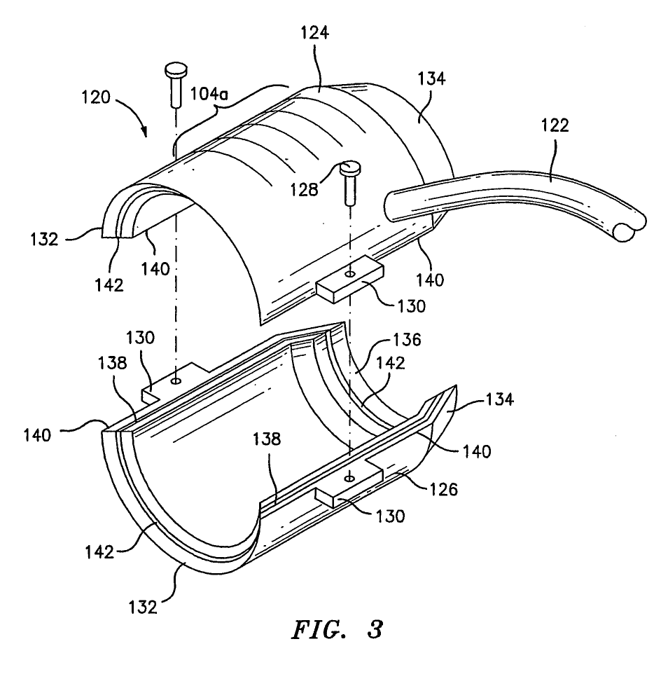

It is envisioned that pusher tube assembly 104 can be either rigid of flexible in order to provide a certain degree of deflection thereof. It is further envisioned, as seen in FIG. 1, that pusher tube assembly 104 and/or vacuum collar 120 (See FIGS. 2 and 3) can be provided with a series of gradations 104a defining a measurement scale on the outer surface thereof. As will be described in greater detail below, gradations 104a are useful in providing the surgeon with a readily ascertainable position of the distal end of cartridge assembly 106 when the distal end of surgical stapling instrument 100 is inserted within the lumen of the body.

Turning now to FIG. 2, an enlarged view of the distal end of surgical stapling instrument 100 is shown. A vacuum collar 120 is operatively coupled to a portion of pusher tube assembly 104 and cartridge assembly 106 such that venting holes 116 (not shown in FIG. 2) are entirely covered by collar 120. A flexible vacuum conduit or tube 122 is operatively and fluidly coupled between collar 120 and a vacuum source 250. In use, as will be described in greater detail below, the vacuum source provides a negative pressure to inner chamber 106a of cartridge assembly 106 via vacuum tube 122, collar 120 and venting holes 116. Vacuum collar 120 can be of any suitable length, shape or breath. Thus it can be axially short to encompass only a cartridge assembly or it can extend along any suitable length of the shaft.

It is contemplated that a passageway ca e formed within or can be formed radially into the inner or outer surface of the wall of the collar.

As seen in detail in FIG. 3, collar 120 preferably includes a pair of cuffs 124, 126 which are adapted to be joined together by any suitable means, for example, by screws 128 threadingly received in a pair of tabs 130. Preferably, cuffs 124, 126 are semi-cylindrical, however, it is envisioned that cuffs 124, 126 can be any shape, such as for example, ovoid, elliptical and the like. Tabs 130 project radially outward from each longitudinal edge 140 of each cuff 124, 126. Each cuff 124, 126 is provided with an angled distal edge or rim surface 132 which substantially corresponds to the angle of conical section 118 (see FIG. 1) of cartridge assembly 106. In addition, each cuff 124, 126 is provided with a conical or angled proximal wall portion 134 terminating in an end surface 136 which is substantially coplanar with a longitudinal axis of collar 120. The angle and the length of proximal wall portion 134 is selected such that when cuffs 124, 126 are joined together, surfaces 136 snuggly contact the outer surface of pusher tube assembly 104.

While a single vacuum tube 122 is shown operatively coupled to cuff 124, it is envisioned that vacuum tube 122 can be operatively coupled to cuff 126 or that a pair or a plurality of vacuum tubes can be provided which are each operatively coupled to a respective cuff 124, 126. As discussed above, vacuum tube 122 interconnects collar 120 and vacuum source 250, e.g., a vacuum pump, which vacuum source 250 provides a vacuum or negative pressure inside collar 120 and subsequently to inner chamber 106a of cartridge assembly 106.

In order to ensure an air tight seal between cuff 124 and cuff 126, one or more sealing elements or seals, e.g., a sealing element 138 is provided along longitudinal edge surface 140 of at least one of cuffs 124, 126. In addition, in order to provide an air tight seal between collar 120 and each of conical section 118 of cartridge assembly 106 and the outer surface of pusher tube assembly 104, a sealing element 142 is provided along angled distal surface 132 and along surface 136.

While tabs 130 and screws 128 have been disclosed for securing or coupling the pair of collars 124, 126 to one another, it is envisioned that alternate coupling mechanisms can be provided for effectuating the coupling or securement, which coupling mechanisms include but are not limited to, for example, a C-clamp or hose clamp disposed along the outer surface of the collars and engaged in a groove formed on the collar; a hinge provided along one longitudinal edge 140 of collars 124, 126, and a locking mechanism provided along the opposite longitudinal edge 140 for securing cuffs 124, 126 to one another, snap fitting members, and other suitable mechanisms or means apparent to one skilled in the art. In addition, while proximal walls 134 are shown as being planar and angled it is appreciated by one skilled in the art that these walls can be orthogonal, rounded or otherwise suitably shaped relative to collar 120.

Turning now to FIG. 4, a partial cross sectional view, taken substantially along the longitudinal axis of a distal end portion of surgical stapling apparatus 100, with vacuum collar 120 in place, is shown. Vacuum collar 120 forms a vacuum chamber 144 around conical section 118. Thus, as a vacuum “V” is applied to collar 120, through vacuum tube 122, air is drawn into annular blade 146, through vent openings 148 formed in a staple pusher 150, through venting holes 116 formed in conical section 118, into vacuum chamber 144 and out through tube 122. Optionally, a filter (not shown) can be provided over venting holes 116 in order to prevent material (e.g., tissue, skin, flesh, blood, etc.) from entering tube 122. A seal 152 is provided between an inner surface of tube assembly 104 and an outer surface of a quill 154, which quill 154 receives an internal shaft portion 156 of anvil assembly 108 therein. Seal 152 ensures that vacuum “V” is created solely as a result of a negative pressure generated through annular blade 146, inner chamber 106a and venting holes 116.

Turning now to FIGS. 5-7, a method of using the above described surgical stapling apparatus 100, in combination with vacuum collar 120, for the removal of rectal mucosal tissue 200 from a human patient is shown and described. As depicted in FIGS. 5-7 the surgical method is being performed on a patient suffering from a severe case of internal hemorrhoids that are enlarged around the entire anal and rectal circumference.

Looking initially at FIG. 5, anvil assembly 108 preferably is approximated to cartridge assembly 106 and is inserted, through the anus 202, into the rectum 204 of the patient such that the distal end of cartridge assembly 106 is located approximately at least at dentate line “D”, preferably above the dentate line and the hemorrhoids “H” and, more preferably about 3-4 cm above dentate line “D”. In the rectal mucosectomy procedure, preferably only rectal mucosal tissue, i.e., no hemorrhoidal tissue, is vacuum drawn into the inner chamber. The depth of insertion of the distal end of cartridge assembly 106 is facilitated by the use of gradations 104a which can be at least 6 or 7 cm gradations provided on the outer surface of pusher tube assembly 104. The gradations are to assist the user to correctly place the distal end of the cartridge assembly at the desired accurate location, preferably above the dentate line. Once suitably located, surgical stapling apparatus 100 is then opened so that the proximal end of anvil assembly 108 is located 2-3 cm from the distal end of cartridge assembly 106. Then, the surgeon activates vacuum source 250 thereby creating vacuum “V” through vacuum chamber 144, venting holes 116, inner chamber 106a and annular blade 146. Vacuum “V” effectively draws rectal mucosal tissue 200 radially inward toward shaft 156 and proximally or axially into inner chamber 106a and annular blade 146.

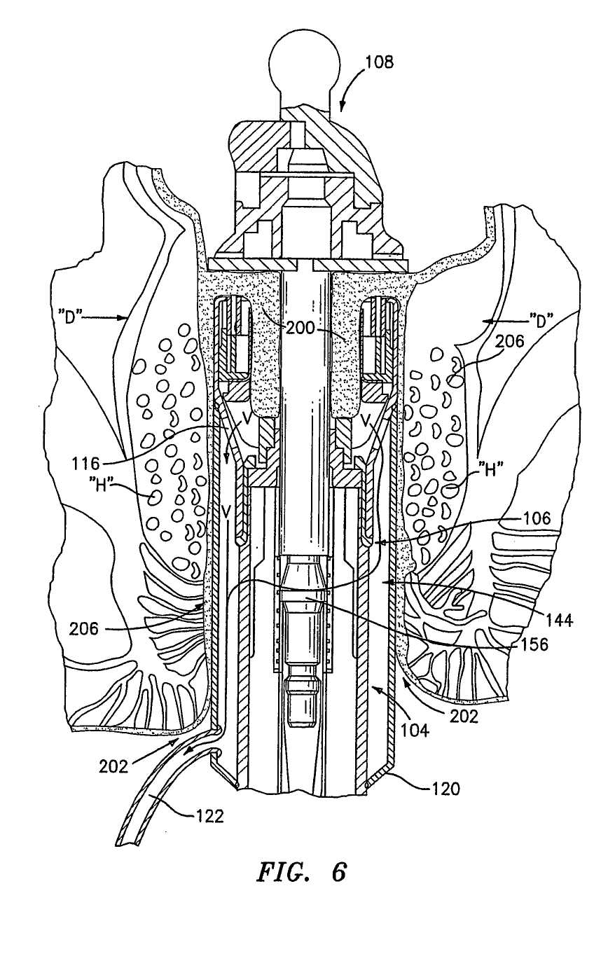

Turning now to FIG. 6, surgical stapling apparatus 100 is shown after the surgeon has rotated wing nut 110 (see FIG. 1) and drawn anvil assembly 108 towards cartridge assembly 106 and closed surgical stapling apparatus 100. During closure, as the space between anvil assembly 108 and cartridge assembly 106 is gradually reduced, the effect of vacuum “V” on rectal mucosal tissue 200 is increased. This draws in increasingly more of rectal mucosal tissue 200 into annular blade 146. As seen in FIG. 6, wing nut 110 has been rotated until anvil assembly 108 is closed onto cartridge assembly 106 thereby securing rectal mucosal tissue 200 within annular blade 146 and between anvil assembly 108 and cartridge assembly 106. The surgeon slowly rotates wing nut 10 an additional amount to ensure that vacuum “V” sufficiently draws all of or as much of rectal mucosal tissue 200 into annular blade 146 as possible under the circumstances. It is important to note that, if wing nut 110 is rotated too quickly, anvil assembly 108 will close too quickly on cartridge assembly 106 and potentially all of the desired rectal mucosal tissue 200 will not be drawn into annular blade 146.

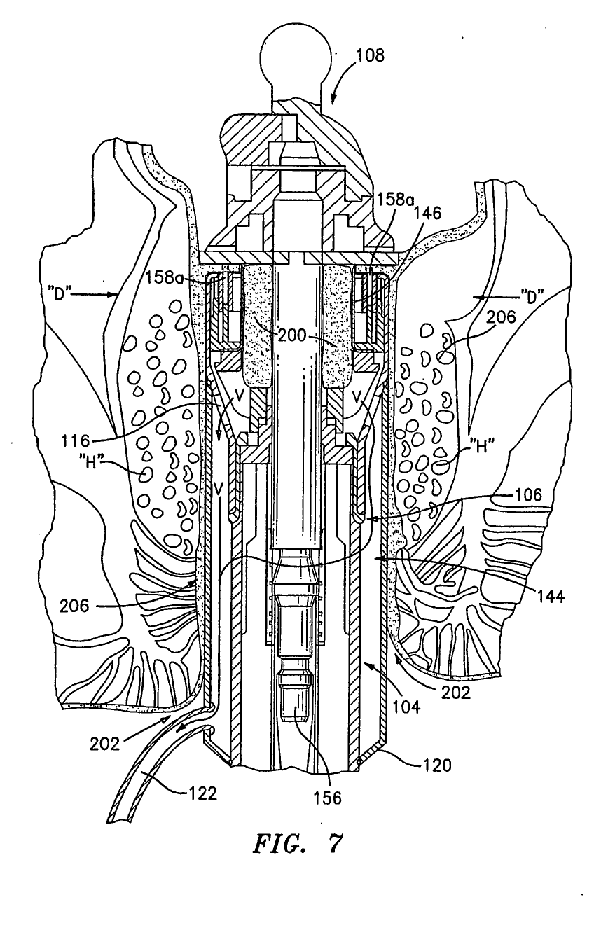

Finally, as seen in FIG. 7, with rectal mucosal tissue 200 substantially completely drawn into annular blade 146, the surgeon fires surgical stapling apparatus 100 by first rotating safety lock 114 (see FIG. 1) and then actuating handles 112 in direction “A” (see FIG. 1). By firing surgical stapling apparatus 100, an annular ring of unformed fasteners or staples 158 (see FIG. 5) is expelled from cartridge assembly 106 into and through the portions of rectal mucosal tissue 200, compressed between anvil assembly 108 and cartridge assembly 106, against the anvil (not shown) of anvil assembly 108, thereby forming staples 158 against the anvil and deforming them from their initial open legged configuration to their formed virtually closed legged configuration into and securing the compressed rectal mucosal tissue 200. Preferably simultaneously with the staple formation, annular blade 146 advances distally against the anvil and severs rectal mucosal tissue 200 from the rectal wall 206 of the patient. Annular blade 146 is positioned radially inward of and adjacent to the formed staples 158a. Annular blade 146 defines an interior chamber 147 (see FIG. 4).

To remove the surgical stapling instrument from the patient, the surgeon first rotates wing nut 110 (FIG. 1), in an opposite direction, in order to space anvil assembly 108 from cartridge assembly 106 thereby releasing the interstitially compressed stapled mucosal tissue of rectal wall 206 and then carefully withdraws the distal end of the surgical stapling instrument from anus 202 of the patient. The cut rectal mucosal tissue, comprising that which is retained in the inner chamber, largely within annular blade 146, is thus removed from the patient. In cases where the rectal mucosal tissue completely surrounds the surgical instrument, a ring or cuff of severed material is disposed about internal shaft portion 156, hence the term “rectal-cuff-mucosectomy”. This is considered to be a mucosal anastomosis.

In an alternative method, the distal end of surgical stapling apparatus 100 is inserted, through the anus 202, into the rectum 204 of the patient while the anvil assembly 108 is opened or open or suitably spaced, preferably about 2 cm, from the distal end of the cartridge assembly 106. Surgical stapling apparatus 100 is then advanced such that the distal end of cartridge assembly 106 is positioned about 2-3 cm beyond dentate line “D”. Once properly positioned, the surgeon activates vacuum source 250 to draw rectal mucosal tissue 200 into the distal end of surgical stapling apparatus 100. After all of rectal mucosal tissue 200 has been drawn into annular blade 146 of stapling assembly 106, wing nut 110 is rotated, in an opposite direction, to thereby close anvil assembly 108 onto cartridge assembly 106 and to secure rectal mucosal tissue 200 within the inner chamber, largely within annular blade 146 and between anvil assembly 108 and cartridge assembly 106. Once rectal mucosal tissue 200 has been sufficiently drawn into annular blade 146, the surgeon fires surgical stapling apparatus 100 as described above in order to fasten the mucosal wall and sever rectal mucosal tissue 200 from the patient.

In either of the two methods described above, it is preferred that vacuum source 250 is activated to produce a vacuum of at least 8-10 bars.

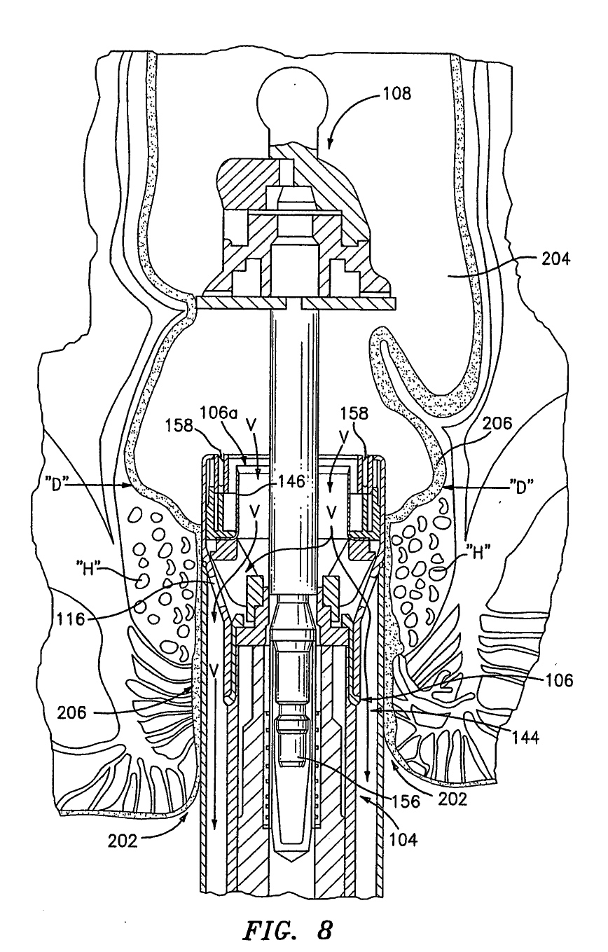

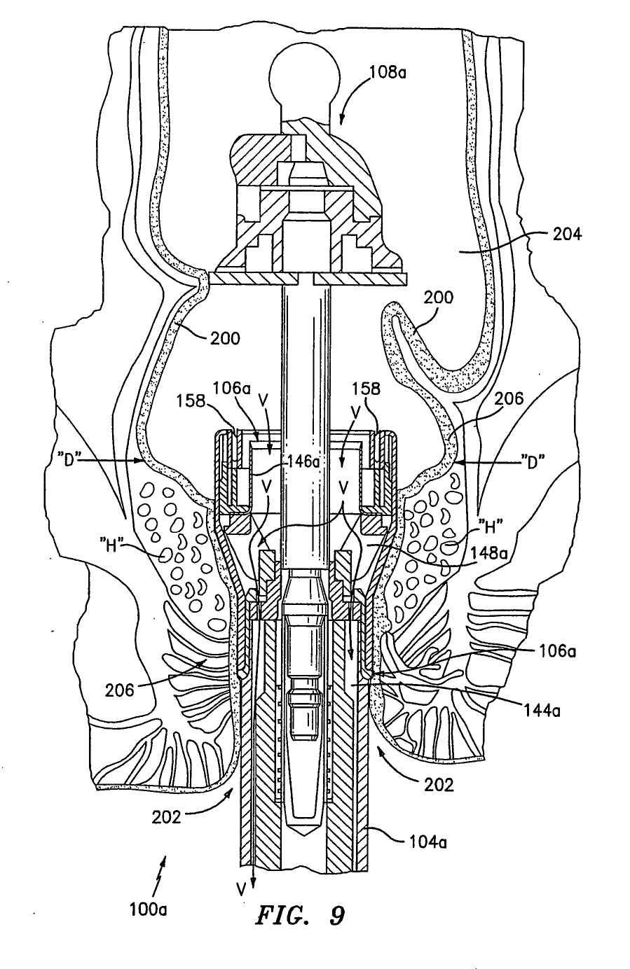

In an alternative embodiment of the surgical stapling apparatus, as seen in FIG. 8, a relatively short vacuum collar 120 of stapling apparatus 100 has been replaced by a longer collar, for example a tube or sheath 152 extending a greater length, for example, substantially the entire length of pusher tube assembly 104. Sheath 152 defines a circumferential lumen 154 through which vacuum “V” is drawn. While a circumferential lumen 154 is disclosed, it is envisioned that one or more less-than-fully circumferential and/or less-than-full length lumen(s), sheaths, passages or channels can be provided externally of or formed radially into and along the outer surface of tube assembly 104, which lumen(s), etc. interconnect venting holes 116 to vacuum source 250 (see FIG. 2). Stapling apparatus 100 is further provided with a valve 252, operatively and fluidly coupled along tube 122 (see FIG. 2). Valve 252 preferably has three positions, a vacuum “ON” position wherein vacuum “V” is drawn in through lumen 154, a vacuum “OFF” position wherein no vacuum “V” is drawn in through lumen 154 and a venting position wherein the area within annular blade 146 is vented.

It is important to note that a stapling apparatus in accordance with the present disclosure preferably has one or more apertures or vent holes formed about the cartridge assembly 106 and/or tube assembly 104, to ensure that the vacuum generated draws the needed air through the opening between the anvil assembly 108 and the cartridge assembly 106 and into inner chamber 106a.

Preferred operation of the stapling apparatus 100, disclosed in FIG. 8, having a valve 252, will now be discussed. With anvil assembly 108 spaced a distance from cartridge assembly 106 and valve 252 in the vacuum “OFF” position, anvil assembly 108 and cartridge assembly 106 are inserted, through the anus 202, into the rectum 204 of the patient such that anvil assembly 108 and a distal end portion of cartridge assembly 106 are located distally of hemorrhoidal tissue “H”. Next, with the vacuum source activated, the surgeon manipulates valve 252 from the vacuum “OFF” position to the vacuum “ON” position thereby creating vacuum “V” through vacuum chamber 144, venting holes 116 and annular blade 146. Vacuum “V” effectively draws rectal mucosal tissue 200 inward toward shaft 156 and proximally or axially into inner chamber 106a and annular blade 146. The surgeon then rotates wing nut 110 (see FIG. 1), drawing anvil assembly 108 towards cartridge assembly 106, until anvil assembly 108 has been closed onto cartridge assembly 106 and rectal mucosal tissue 200 has been mechanically secured so that it extends within the chamber and preferably into the interior chamber defined by annular blade 146. With hemorrhoid 200 secured in place, the surgeon next manipulates valve 252 from the vacuum “ON” position to the venting position. By so doing, the vacuum “V” being drawn through annular blade 146 is removed and the area within annular blade 146 is allowed to substantially return to atmospheric pressure.

The surgeon then fires surgical stapling apparatus 100 by first rotating safety lock 114 (see FIG. 1) and then actuating handles 112 in direction “A”. Firing of surgical stapling apparatus 100 effectively expels an annular ring of unformed staples 158 (see FIG. 5) from cartridge assembly 106 into and through that portion of rectal mucosal tissue 200 compressed between anvil assembly 108 and cartridge assembly 106 and against the anvil (not shown) of anvil assembly 108. Staples 158 are thus deformed, against the anvil, from their initial open legged configuration to their formed virtually closed legged configuration thus securing the compressed rectal mucosal tissue 200. Preferably, simultaneously with the staple formation, annular blade 146 severs rectal mucosal tissue 200 from the rectal wall 206 of the patient, radially inward of and adjacent to the formed staples 158a (see FIG. 7) thus completing the mucosal anastomosis. The surgeon then completes the remainder of the surgical procedure in the manner previously discussed.

Each of the above described methods according to the present disclosure leaves the patient with rectal mucosal tissue 200 removed and a hemostatic ring of formed staples at the site from whence rectal mucosal tissue 200 was removed. It can be seen that the apparatus of the disclosure can be employed to effect mucosal anastomosis, full or partial, as well as other anastomoses procedures.

By eliminating the need for suturing, the complexity and time for performing a rectal mucosectomey is reduced. The trauma inflicted on the patient accordingly is also reduced. The use of a vacuum to radially and axially draw in, for example, rectal mucosal tissue into the distal end of a circular surgical stapling apparatus enables a surgeon to perform, for example, a rectal mucosectomy in a far more efficient manner with less internal manual activity by eliminating the need of undertaking the difficult and time consuming procedure of suturing required by the prior art methods. In effect, a surgeon using the apparatus and method according to the present disclosure is capable of aiding more patients by being able to perform more rectal mucosectomies in a given period of time as compared to a surgeon using conventional instruments and procedures.

An advantage of the rectal mucosectomy procedures described herein is that often hemorrhoids can be treated without removing them. By removing rectal mucosal tissue above the hemorrhoids, branch arteries within the tissue that supply blood to the hemorrhoids are severed. The reduction in blood supply to the hemorrhoids causes them to shrink and be preserved in and less problematical to the patient.

It is of course envisioned that the vacuum application apparatus and method of the disclosure can be employed in combination with a suturing procedure that, for example, approximates rectal mucosal tissue by use of a suture, usually a purse string suture.

As discussed above, while the present apparatus and method has been described in detail mainly with regard to performing a rectal mucosectomy, it is envisioned and it will be apparent that a surgical apparatus and a method in accordance with the present disclosure can be used in connection with other surgical procedures as well. The above disclosure should therefore not be construed as limiting, but merely as exemplifications of preferred embodiments.

Turning now to FIG. 9, a surgical stapling apparatus, according to an alternate embodiment of the disclosure, is shown generally as 100a. Unlike the embodiment of surgical stapling apparatus 100 shown in FIGS. 1-8, the embodiment of FIG. 9 does not include venting holes 116 provided in conical section 118 of cartridge assembly 106.

According to the embodiment shown in FIG. 9, a vacuum channel or chamber 144a is defined circumferentially around the inside of tube assembly 104a, preferably between an inner surface of tube assembly 104a and an outer surface of actuating assembly 107a. Vacuum chamber 144a extends along the length of tube assembly 104a and is in fluid communication with inner chamber 106a and with a vacuum port (not shown), which vacuum port is used to fluidly couple vacuum chamber 144a of surgical stapling apparatus 100a to vacuum source 250 (See FIG. 1).

Thus, when the surgeon activates vacuum source 250 to apply vacuum “V” to the operative site, vacuum “V” is drawn through a vacuum channel composed of vacuum chamber 144a, vent openings 148a and, preferably through annular blade 146a and inner chamber 106a. Accordingly, vacuum “V” effectively draws rectal mucosal tissue 200 into inner chamber 106a, largely into the interior chamber of annular blade 146.

It is envisioned that each of surgical stapling apparatus 100 and 100a (for simplicity, hereinafter referred to as surgical stapling apparatus 100) is provided with an annular blade 146 having a flange extending outwardly circumferentially around the periphery of annular blade 146, for securing annular blade 146 within surgical stapling apparatus 100. The bottom of annular blade 146 is desirably entirely or largely open to receive more rectal mucosal tissue. It can have spokes extending to a central hub, or it can have a bottom wall (not shown) extending across its bottom portion. The bottom wall preferably has a central aperture configured and adapted to permit shaft portion 156 to pass therethrough and one or more openings of desired location, shape and size to pass sufficient vacuum therethrough. Preferably, annular blade is provided with a O-ring type seal disposed within the central aperture in order to create a fluid tight seal between annular blade 146 and shaft portion 156.

Those skilled in the art will envision other modifications within the scope and spirit of the present disclosure.

What is claimed is:

1. A surgical instrument for performing a rectal mucosectomy comprising:
a shaft having a proximal end and a distal end;
a cartridge assembly including a plurality of fasteners and operatively coupled to the distal end of the shaft, the cartridge assembly having a housing and a distal end, and defining an inner chamber;
at least one opening formed in the surgical instrument, the opening extending through the exterior of the instrument and being in communication with the inner chamber and adapted to communicate with a vacuum source;
an anvil assembly operatively coupled to the cartridge assembly and disposed opposite the cartridge assembly, the cartridge assembly and the anvil assembly being configured and adapted to fire and secure fasteners into target rectal mucosal tissue; and
a vacuum channel extending through the cartridge assembly and in communication with the inner chamber such that when a vacuum is applied through the channel, the vacuum is transmitted into the inner chamber of the cartridge assembly to draw rectal mucosal tissue into the inner chamber.

2. The surgical instrument according to claim 1, wherein the at least one opening is in the exterior of the housing.

3. The surgical instrument according to claim 1, wherein the at least one opening is located proximally of the cartridge assembly.

4. The surgical instrument according to claim 1, wherein the at least one opening extends from the shaft to the inner chamber.

5. The surgical instrument according to claim 1, wherein the vacuum is drawn into the distal end of the cartridge assembly.

6. The surgical instrument according to claim 1, wherein the vacuum channel transmits the vacuum through the at least one opening to draw rectal mucosal tissue from areas surrounding and/or above the distal end of the cartridge assembly axially into the inner chamber of the cartridge assembly.

7. The surgical instrument according to claim 1, wherein the cartridge assembly includes an annular blade disposed therewithin, radially inward of the fasteners.

8. The surgical instrument according to claim 7, wherein the annular blade includes a peripheral wall and an interior chamber radially inward of the wall, wherein the vacuum is transmitted to an area within the interior chamber of the annular blade to draw tissue surrounding and/or above the distal end of the cartridge assembly into the interior chamber.

9. The surgical instrument according to claim 7, wherein the annular blade has a distal end and an open proximal end, and the vacuum is transmitted axially through the proximal end.

10. The surgical instrument according to claim 7, wherein the annular blade has a distal end, a proximal end, and has a bottom wall at its proximal end, the bottom wall having at least one opening therethrough for drawing of the vacuum axially therethrough.

11. The surgical instrument according to claim 1, wherein the vacuum channel extends within and along a length of the shaft.

12. The surgical instrument according to claim 1, wherein the outer surface of the shaft includes gradations arranged axially along a length thereof to allow a user to visually determine an approximate distance from the distal end of the cartridge assembly to the anal ring of the patient when the distal end of the cartridge assembly is inserted into the anal ring of the patient.

13. The surgical instrument according to claim 12, the gradations are sufficient to determine a distance of at least about 5 to 6 cm from the distal end of the cartridge assembly to the anal ring of the patient.

14. A method of performing a rectal mucosectomy comprising the steps of:
providing a circular fastener apparatus having a distal end and including a shaft, a cartridge assembly operatively coupled to the distal end of the shaft and having a plurality of fasteners, an inner chamber, an anvil assembly operatively coupled to the cartridge assembly and supported adjacent the distal end of the shaft adjacent the cartridge assembly, and a vacuum application element configured and adapted to transmit a vacuum to the inner chamber of the cartridge assembly of the circular fastener apparatus;
inserting a distal end of the circular fastener apparatus distally into the rectum of a patient such that the distal end of the cartridge assembly is located at or distally of hemorrhoidal tissue;
providing a gap between the upper border of the cartridge assembly and anvil assembly;
applying a vacuum to the cartridge assembly such that rectal mucosal tissue is drawn into the inner chamber of the cartridge assembly;
approximating the anvil and cartridge assemblies to one another; and
firing the circular fastener apparatus to sever the rectal mucosal tissue from the rectal wall of the patient.

15. The method according to claim 14, wherein the distal end of the cartridge assembly is positioned distally of the hemorrhoidal tissue prior to the application of the vacuum.

16. The method according to claim 14, wherein the anvil and cartridge assemblies are approximated with one another when the circular fastener apparatus is inserted into the rectum, and they are separated from one another prior to application of the vacuum.

17. The method according to claim 14, wherein the distal end of the circular fastener apparatus is inserted into the rectum of the patient such that the distal end of the cartridge assembly is positioned about 3 to 4 cm distally of the dentate line.

18. The method according to claim 14, wherein the anvil assembly is spaced from the cartridge assembly about 2 to 3 cm prior to application of the vacuum.

19. The method according to claim 14, wherein the vacuum is drawn axially through the cartridge assembly.

20. A circular stapler comprising:
a shaft having a proximal and a distal end;
a cartridge assembly having a plurality of staples and operatively coupled to the distal end of the shaft, the cartridge assembly having a housing and a distal end defining an inner chamber;
an anvil assembly operatively coupled to the cartridge assembly; and
a channel adapted to communicate with a source of vacuum and the cartridge assembly to transmit a vacuum to the inner chamber of the cartridge assembly for drawing tissue into the inner chamber of the cartridge assembly.

21. The surgical apparatus according to claim 20, wherein the cartridge assembly includes an annular blade disposed within the inner chamber thereof.

22. The surgical apparatus according to claim 21, wherein the vacuum is transmitted internally of the annular blade.

23. The surgical apparatus according to claim 21, wherein the annular blade has an interior chamber and is disposed radially inside of the fasteners, such that the vacuum draws surrounding tissue into interior chamber of the annular blade.

24. A circular stapler comprising:
a shaft having a proximal end and a distal end;
a cartridge assembly including a plurality of staples and operatively coupled to the distal end of the shaft, the cartridge assembly including at least one opening and defining an inner chamber, the at least one opening extending from the exterior of the apparatus through to the inner chamber;
an anvil assembly supported adjacent the cartridge assembly and movable relative to the cartridge assembly between spaced and approximated positions; and
a vacuum collar configured and adapted to be couplable to at least the cartridge assembly such that the at least one opening formed in the cartridge assembly is covered by the vacuum collar, such that when the vacuum collar is applied to at least a portion of the cartridge assembly, and a vacuum is applied to the vacuum collar, the vacuum is transmitted through the at least one opening to the inner chamber of the cartridge assembly to draw tissue into a gap defined between the cartridge and anvil assemblies.

25. The surgical apparatus according to claim 24, wherein the vacuum collar creates a fluid tight seal around the cartridge assembly and the shaft.

26. The surgical apparatus according to claim 24, wherein the vacuum collar defines a vacuum chamber around at least a portion of the shaft and of the cartridge assembly.

27. The surgical apparatus according to claim 24, wherein the vacuum collar includes at least one vacuum transmitting lumen for transmitting the vacuum to the vacuum chamber.

28. The surgical apparatus according to claim 24, wherein the vacuum collar includes a pair of semi-cylindrical cuffs which are couplable to one another by fastening elements.

29. The surgical apparatus according to claim 28, wherein the vacuum collar transmits the vacuum through the at least one opening formed in the cartridge assembly to draw tissue surrounding and distally of a distal end of the cartridge assembly into the inner chamber of the cartridge assembly.

30. The surgical apparatus according to claim 24, wherein the cartridge assembly includes an annular blade located radially inward of the fasteners.

31. The surgical apparatus according to claim 30, wherein the vacuum is transmitted to an area within the inner chamber to draw tissue surrounding and/or above the distal end of the cartridge assembly into the inner chamber.

32. The surgical apparatus according to claim 24, further including a source of vacuum fluidly and operatively coupled to the vacuum collar.

33. The surgical apparatus according to claim 24, further including a valve fluidly coupled to the source of vacuum via a conduit wherein the valve controls the transmission of vacuum to the vacuum collar.

34. The surgical apparatus according to claim 24, wherein the vacuum chamber extends substantially along the entire length of the shaft.

35. A vacuum collar for use with a circular stapler of the type including an elongated shaft having a distal end supported adjacent the cartridge assembly and coupled to a distal end of the shaft and an anvil assembly supported adjacent the cartridge assembly, the vacuum collar comprising:
a pair of cuffs operatively couplable to one another, each of the pair of cuffs being configured and adapted, when coupled to the circular stapler, to surround at least a portion of the shaft and/or the cartridge assembly and to provide a passageway to the cartridge assembly; and
at least one connection for a vacuum conduit to operatively and fluidly couple the conduit to at least one of the pair of cuffs, the at least one vacuum conduit being for transmitting a vacuum to the vacuum collar and through the passageway to draw a vacuum within the cartridge assembly.

36. The vacuum collar according to claim 35, wherein the vacuum collar defines a vacuum chamber surrounding at least a portion of the shaft and/or the cartridge assembly when the vacuum collar is coupled to the circular stapler.

37. The vacuum collar according to claim 35, wherein each cuff is semi-cylindrical and includes a pair of longitudinal terminal edges, a distal edge configured and dimensioned to contact and seal an outer surface of the cartridge assembly, and a proximal edge configured and dimensioned to contact and seal an outer surface of the shaft.

38. The vacuum collar according to claim 37, wherein the longitudinal, distal and proximal edges of each cuff includes a sealing element.

39. The vacuum collar according to claim 37, wherein each longitudinal terminal edge includes a tab extending radially outward therefrom, each tab including an aperture formed therein for receiving a fastening element therethrough, wherein the tabs from one cuff align with the tabs from the other cuff.

40. The vacuum collar according to claim 35, wherein the vacuum collar extends along substantially the entire length of the shaft.

41. A method of performing a rectal mucosectomy comprising the steps of:
providing a circular stapler including a shaft having a distal end, a cartridge assembly having a plurality of staples, an inner chamber and a cutting blade, and operatively coupled to the distal end of the shaft, an anvil assembly operatively coupled to the cartridge assembly and supported adjacent to the cartridge assembly, and a vacuum application element configured and adapted to transmit a vacuum to the inner chamber of the cartridge assembly of the circular stapler;
inserting a distal end of the circular stapler into the rectum of a patient such that a portion of the cartridge assembly is located above the dentate line;
applying a vacuum through the application element to the inner chamber of the to the cartridge assembly, such that rectal mucosal tissue is drawn into the inner chamber of the cartridge assembly;
approximating the anvil and cartridge assemblies to one another; and
firing the circular stapler to sever the rectal mucosal tissue from the patient and anastomose the remaining tissue.

42. The method according to claim 41, wherein the distal end of the cartridge assembly is positioned proximally of and/or above the dentate line prior to the application of the vacuum.

43. The method according to claim 41, wherein the anvil and cartridge assemblies are approximated with one another for insertion into the rectum and separated from one another for application of the vacuum.

44. The method according to claim 40, wherein the vacuum application element is a vacuum channel.

45. The method according to claim 40, wherein the vacuum application element is a vacuum collar.

46. A circular stapler for performing a rectal mucosectomy comprising:
a shaft having a proximal and a distal end;
a cartridge assembly containing a plurality of staples and operatively coupled to the distal end of the shaft and defining an inner chamber therein;
an anvil assembly operatively coupled to the cartridge assembly, the cartridge assembly and the anvil assembly being configured and adapted to fire and secure a plurality of staples into a target site; and
a vacuum channel operatively and fluidly couplable to the cartridge assembly to transmit a vacuum to the inner chamber of the cartridge assembly, independent of the anvil assembly, for drawing distal and/or surrounding tissue into the inner chamber of the cartridge assembly.

47. The surgical apparatus according to claim 46, wherein the cartridge assembly includes an annular blade disposed within the inner chamber thereof.

48. The surgical apparatus according to claim 47, wherein the vacuum is transmitted internally of the annular blade.

49. The surgical apparatus according to claim 47, wherein the annular blade defines an interior chamber and is disposed radially inside of the fasteners, whereby the vacuum draws distal and/or surrounding tissue into the interior chamber of the annular blade.

50. A circular stapler for performing a surgical procedure comprising:
a shaft having a proximal end and a distal end;
an anvil retainer assembly;
a circular cartridge assembly including a plurality of staples and operatively coupled to the distal end of the shaft, the cartridge assembly having a housing and a distal end, and defining an inner chamber;
an anvil assembly operatively coupled to the cartridge assembly and disposed opposite the cartridge assembly, the cartridge assembly and the anvil assembly being configured and adapted to fire and secure staples into target tissue; and
a vacuum channel extending externally of the anvil retainer assembly, through the cartridge assembly, and in communication through the channel, a vacuum is transmitted through the channel axially into the inner chamber of the cartridge assembly to draw tissue into the inner chamber.

51. The surgical instrument according to claim 50, wherein the surgical procedure is a circular stapler anopexy.

52. The surgical instrument according to claim 50, wherein the surgical procedure is a hemorrhoidectomy.

53. The surgical instrument according to claim 50, wherein the surgical procedure is a rectal mucosectomey.

54. The surgical instrument according to claim 50, wherein the surgical procedure is an anastomosis.

55. The method according to claim 14, wherein the vacuum is applied at a pressure of at least 8-10 bars.

56. The method according to claim 41, wherein the vacuum is applied at a pressure of at least 8-10 bars.
